SEQUENCE LISTING (16 sequences)

SEQ ID NO: 1 - DNA, 579 bp, Streptococcus agalactiae
atgaaagtgaaaaataagattttaacgatggtagcacttactgtcttaacatgtgctacttattcatcaatcggttatgctgatacaagtgataagaatactgacacgagtgtcgtgactacgaccttatctgaggagaaaagatcagatgaactagaccagtctagtactggttcttcttctgaaaatgaatcgagttcatcaagtgaaccagaaacaaatccgtcaactaatccacctacaacagaaccatcgcaaccctcacctagtgaagagaacaagcctgatggtagaacgaagacagaaattggcaataataaggatatttctagtggaacaaaagtattaatttcagaagatagtattaagaattttagtaaagcaagtagtgatcaagaagaagtggatcgcgatgaatcatcatcttcaaaagcaaatgatgggaaaaaaggccacagtaagcctaaaaaggaacttcctaaaacaggagatagccactcagatactgtaatagcatctacgggagggattattctgttatcattaagtttttacaataagaaaatgaaactttattaa

SEQ ID NO: 2 - DNA, 768 bp, Streptococcus agalactiae
atgaacgttacacatatgatgtatctatctggaactctagtggctggtgcattgttattttcaccagctgtattagaagtacatgctgatcaagtgacaactccacaagtggtaaatcatgtaaatagtaataatcaagcccagcaaatggctcaaaagcttgatcaagatagcattcagttgagaaatatcaaagataatgttcagggaacagattatgaaaaaccggttaatgaggctattactagcgtggaaaaattaaagacttcattgcgtgccaaccctgagacagtttatgatttgaattctattggtagtcgtgtagaagccttaacagatgtgattgaagcaatcactttttcaactcaacatttaacaaataaggttagtcaagcaaatattgatatgggatttgggataactaagctagttattcgcattttagatccatttgcttcagttgattcaattaaagctcaagttaacgatgtaaaggcattagaacaaaaagttttaacttatcctgatttaaaaccaactgatagagctaccatctatacaaaatcaaaacttgataaggaaatctggaatacacgctttactagagataaaaaagtacttaacgtcaaagaatttaaagtttacaatactttaaataaagcaatcacacatgctgttggagttcagttgaatccaaatgttacggtacaacaagttgatcaagagattgtaacattacaagcagcacttcaaacagcattaaaataa

SEQ ID NO: 3 - DNA, 24 bp, Artificial
  Primer
acgagtgtcgtgactacgacctta

SEQ ID NO: 4 - DNA, 24 bp, Artificial
  Primer
tctgtcttcgttctaccatcaggc

SEQ ID NO: 5 - DNA, 26 bp, Artificial
  Primer
acctacaacagaaccatcgcaaccct

SEQ ID NO: 6 - DNA, 25 bp, Artificial
  Primer
tgaggctattactagcgtggaaaaa

SEQ ID NO: 7 - DNA, 26 bp, Artificial
  Primer
catctgttaaggcttctacacgacta

SEQ ID NO: 8 - DNA, 29 bp, Artificial
  Primer
acttcattgcgtgccaaccctgagacagt

SEQ ID NO: 9 - DNA, 150 bp, Arabidopsis thaliana
ggatggtgacatgacaaacggcaaggctttttaggtgatttatttaaaacggcaactcgttttgttgtaggtcgtttctctcttttaagattggacgcctcgccgtttcttttattttctagcttattgtagttgtccatgcttgcttaa

SEQ ID NO: 10 - DNA, 57 bp, Artificial
  Primer
tatactcgagtgaggctattactagcgtggaaaaaggatggtgacatgacaaacggc

SEQ ID NO: 11 - DNA, 65 bp, Artificial
  Primer
tataggatcccatctgttaaggcttctacacgactattaagcaagcatggacaactacaataagc

SEQ ID NO: 12 - DNA, 213 bp, Artificial
  Control Sequence
ctcgagtgaggctattactagcgtggaaaaaggatggtgacatgacaaacggcaaggctttttaggtgatttatttaaaacggcaactcgttttgttgtaggtcgtttctctcttttaagattggacgcctcgccgtttcttttattttctagcttattgtagttgtccatgcttgcttaatagtcgtgtagaagccttaacagatgggatcc

SEQ ID NO: 13 - DNA, 26 bp, Artificial
  Internal control probe
tggtgacatgacaaacggcaaggctt

SEQ ID NO: 14 - DNA, 210 bp, Artificial
  Positive internal control
ctcgagacgagtgtcgtgactacgaccttaggatggtgacatgacaaacggcaaggctttttaggtgatttatttaaaacggcaactcgttttgttgtaggtcgtttctctcttttaagattggacgcctcgccgtttcttttattttctagcttattgtagttgtccatgcttgcttaagcctgatggtagaacgaagacagaggatcc

SEQ ID NO: 15 - DNA, 56 bp, Artificial
  Primer
tatactcgagacgagtgtcgtgactacgaccttaggatggtgacatgacaaacggc

SEQ ID NO: 16 - DNA, 63 bp, Artificial
  Primer
tataggatcctctgtcttcgttctaccatcaggcttaagcaagcatggacaactacaataagc

(19) United States
(12) Patent Application Publication
(10) Pub. No.: US 2006/0088849 A1
(43) Pub. Date: Apr. 27, 2006

(54) Compositions and methods for the diagnosis of group B streptococcus infection

(75) Inventor: Scott B. Happe, Austin, TX (US)

Correspondence Address: PALMER & DODGE, LLP;KATHLEEN M. WILLIAMS / STR, 111 HUNTINGTON AVENUE, BOSTON, MA, 02199, US

(73) Assignee: Stratagene California

(21) Appl. No.: 11/139,257

(22) Filed: May 27, 2005

Related U.S. Application Data
(60) Provisional application No. 60/575,124, filed on May 28, 2004.

(51) Int. Cl. C12Q 1/68; C07H 21/04

(52) U.S. Cl. 435/6; 536/24.1

(57) ABSTRACT

The present invention relates to methods of detecting a Group B Streptococcus (GBS) bacterium in a sample. In particular, the present invention provides compositions, kits and methods for detecting the gbs1539 gene of a GBS bacterium.

### RELATED APPLICATION(S)

This application claims the benefit of U.S. Provisional Application No. 60/575,124 filed on May 28, 2004. The entire teachings of the above application is incorporated herein by reference.

### FIELD OF THE INVENTION

The present invention relates to compositions and methods for the detection and diagnosis of Group B Streptococcus (GBS).

### BACKGROUND

Group B Streptococcus (GBS) colonizes the gastrointestinal and genitourinary tracts of humans. While in most cases infection with this organism does not cause disease in healthy adults, GBS is the predominant cause of neonatal sepsis, meningitis, and pneumonia (Baker, et al. (1973), J Pediatr. 82:724-9; Barton, et al. (1973), J. Pediatr. 82:719-23; Franciosi, et al. (1973), J. Pediatr. 82:707-18; McCraken (1973), J. Pediatr. 82:703-6). Bacteria are most often transferred from an asymptomatic mother to child during passage through the birth canal. An average of about 25% of pregnant women in the United States are colonized by GBS at the time of delivery (Ke and Bergeron (2001), Expert Rev Mol Diagn 1:175-181; Schuchat (1998), Clin Microbiol Rev 11 :497-513; Platt and O'Brien (2003), Obstet Gynecol Sunv 58:1 91-196). Due to the intermittent nature of GBS colonization, it is recommended by the Centers for Disease Control that pregnant women be screened between 35-37 weeks' gestation to determine colonization status near the time of delivery (Centers for Disease Control and Prevention, 2002), so that appropriate antibiotic therapy can be administered prior to labor. The standard method for screening involves collection of vaginal and rectal swabs, followed by culture of the organism on selective media. Culture identification can be confirmed by a variety of techniques including biochemical assays, probe hybridization, and antigen-based tests.

Coventional culture methods require up to 36 hours to obtain results and predict only 87% of women likely to be colonized by GBS at delivery. A rapid, sensitive, and specific test for detection of GBS directly from clinical specimens would allow for a simpler and more efficient prevention program. Both gel-based and real-time PCR assays have been described that provide such rapid results (Bergeron et al., 2000; Ke et al., 2000). Currently, only one commercial real-time PCR assay is sold by Cepheid under the tradename IDI-Strep B which is based on the original assay developed by Bergeron, Ke, and colleagues. Briefly, bacterial cells are eluted from swab specimens, the DNA content is released from the cells by glass bead lysis, and the sample is combined with reaction buffer for rapid thermal cycling. The assay specifically targets the CAMP-factor (cfb) gene of GBS. CAMP-factor is an extracellular protein that acts synergistically with Staphylococcus aureus β-toxin to produce a zone of clearance on sheep blood agar (Christie, et. al. (1944), Aust J Exp Biol Med Sci. 22:197-200; Jurgens, et al. (1985), J Chromatogr. 348:363-370). This phenomenon is the basis for a biochemical test to identify cultured bacterial cells that are suspected to be GBS (Wilkinson (1977), J CI1n Microbiol. 6:42-5). Virtually all GBS isolates have been shown to produce CAMP-factor (Podbielski et al. (1994), Med Microbiol Immunol. 183:239-56). U.S. Patent application No. 2003/0207273A1 by Bett Wu et al. specifically indicates CAMP-factor as a diagnostic target sequence for detection of GBS. U.S. Pat. No. 6,004,754 discloses a newly identified DNA sequence from GBS (i.e., the gbs3.1 DNA) and the use of this DNA for GBS detection. U.S. patent application No. 2003/0049636A1 by Bergeron et al. describe methods of using antibiotic resistance genes for the detection of a variety of bacteria.

### SUMMARY OF THE INVENTION

The present invention provides probes, primers and methods for a rapid, sensitive, specific, user friendly and reliable detection of GBS.

The present invention provide a method of detecting the presence of a Group B Streptococcus (GBS bacterium) in a sample, comprising: (a) contacting the sample with a primer which hybridizes to the sequence of SEQ ID NO:1 or its complementary sequence thereof under conditions permitting the production of an extension product from the primer; and (b) detecting the presence of the extension product, where the presence of the extension product is indicative of the presence of a GSB bacterium in the sample.

In one embodiment, the extension product is produced by a polymerase chain reaction (PCR).

In another embodiment, the primer comprises a sequence of SEQ ID NO:3 or SEQ ID NO:4 or a complementary sequence thereof.

In another embodiment, step (a) of the subject method comprises contacting the sample with a pair of primers, where at least one primer comprises a sequence of SEQ ID NO:3 or SEQ ID NO:4 or a complementary sequence thereof.

In another embodiment, the subject method further comprises a labeled probe in step (a), where the probe hybridizes to the extension product and the hybridization generates a detectable signal which is indicative of the presence of a GSB bacterium in the sample.

In one embodiment, the labeled probe comprises a sequence of SEQ ID NO:5 or a complementary sequence thereof.

In another embodiment, the probe is labeled with a detectable label and the hybridization generates a detectable signal which is indicative of the presence of a GBS bacterium in the sample.

Preferably, the detectable label is a fluorescent label.

In one embodiment, the sample is obtained from an individual suspected of being infected with GBS.

The present invention provides an isolated oligonucleotide comprising a sequence selected from the group consisting of SEQ ID NOs. 3-5 and their complementary sequences thereof.

The present invention further provides an isolated polynucleotide comprising a sequence of SEQ ID NO:9 or SEQ ID NO:14.

The present invention provides a pair of isolated oligonucleotides comprising a first oligonucleotide and a second oligonucleotide, where the first oligonucleotide comprises the sequence of SEQ ID NO:3 or its complementary sequence thereof and the second oligonucleotide comprises the sequence of SEQ ID NO:4 or its complementary sequence thereof.

The present invention also provides a composition comprising an oligonucleotide comprising a sequence selected from the group consisting of SEQ ID NOs. 3-5 and their complementary sequences thereof.

In one embodiment, the isolated oligonucleotide is 8-100 nucleotides in length.

Preferably, the oligonucleotide is 15-50 nucleotides in length.

In one embodiment, the subject composition of the present invention further comprises a reagent selected from the group consisting of: a DNA polymerase, a control DNA, a control primer, and a deoxynucleotide triphosphate (dNTP).

The present invention provides a kit comprising an oligonucleotide comprising a sequence selected from the group consisting of SEQ ID NOs. 3-5 and their complementary sequences thereof, and packing materials therefore.

The subject kit of the present invention may further comprise a reagent selected from the group consisting of a DNA polymerase, a control DNA, a control primer, and a dNTP.

Other advantages, objects, features and embodiments of the present invention will become apparent from the detailed description which follows.

### BRIEF DESCRIPTION OF THE DRAWINGS

FIG. 1 shows GBS genomic DNA was titrated (in duplicate) into the QPCR assay containing gbs1539 primer/probe set according to one embodiment of the invention.

FIG. 2 shows GBS genomic DNA was titrated (in duplicate) into the QPCR assay containing CAMP-factor primer/probe set according to one embodiment of the invention.

FIG. 3 shows the Ct values of each sample in FIGS. 1 and 2 were plotted against the log of input genomic DNA copy number according to one embodiment of the invention.

FIG. 4 shows GBS genomic DNA was titrated in serial dilutions of 1:2 from 160 copies to 10 copies, and tested in either the gbs1539 assay (bottom, gray line) or the CAMP-factor assay (top, black line) according to one embodiment of the invention.

FIG. 5 shows GBS genomic DNA was titrated from 5×10⁵ to 10 copies in the gbs1539 assay using the Mx3000p instrument according to one embodiment of the invention.

FIG. 6 shows plasmid containing the cloned CAMP-factor internal control sequence (SEQ ID NO:12) was titrated from 5×10⁵ (left most blue line) to 50 copies (right most gold yellow line) in the GBS QPCR assay, and signal was detected using the HEX emission channel according to one embodiment of the invention.

FIG. 7 shows GBS genomic DNA was titrated from 5 to 5×10⁴ copies in the standard CAMP-factor QPCR assay containing 100 copies of internal control template and 200 nM HEX-labeled internal control probe according to one embodiment of the invention.

### DETAILED DESCRIPTION

Definitions

The present invention provides PCR diagnostic assays and kits for detecting the presence of Group B Streptococcus bacteria in a sample. The subject methods can be designed by choosing a bacterial diagnostic target nucleotide sequence based upon its lack of homology to other bacterial sequences in public databases and its likelihood to be conserved among GBS isolates due to its surface localization. In one embodiment, the bacterial diagnostic target encodes a cell wall protein, such as the exemplified gbs1539 protein, which contains an LPxTG motif required for anchoring of proteins to the cell wall of gram-positive bacteria. The gbs1539 target has a clear advantage over other available targets, e.g., the CAMP-factor target, as the signal can be obtained with fewer thermocycles and is more reproducible at lower copy numbers. Therefore, overall cycling time is reduced and diagnosis is more sensitive than with existing PCR-based assays. The PCR methods described herein allow cycling times that are dramatically reduced compared to other assays using conventional block thermocyclers. For example, using the gbs1539 primer set, 10 copies of GBS DNA can be detected in 46 minutes using the real-time PCR instrument sold by Stratagene under the tradename Mx3000p, as compared to a mean of 25 copies and a 1-hour cycle time using conventional instrumentation.

Unless defined otherwise, all technical and scientific terms used herein have the same meaning as commonly understood by one of ordinary skill in the art to which this invention belongs.

“Group B Streptococcus (GBS),” as used herein, refers to any bacterial species of the genus Streptococcus that present the Lancefield group B antigen on the surface. Group B Streptococci belong to one of nine serotypes based upon the type of capsular polysaccharide that is synthesized from the cps gene cluster. The term GBS incluses, but is not limited to, all known isolates of Streptococcus agalactiae and includes any unknown isolates that might be identified in the future. Examples of GBS strains include, but are not limited to GBS Ia (ATCC 12400), GBSIb (ATCC 12401), GBS Ic (ATCC 25941), GBS II (ATCC 12973), and GBS III (ATCC 12403), as well as those described in U.S. Pat. No. 6,004,754 (hereby incorporated in its entirety by reference).

As used herein, “sample” refers to any substance containing or presumed to contain a nucleic acid of interest, for example a target nucleic acid sequence such as the exemplified gbs1539 gene found in a Group B Streptococcus (GBS) bacterium, or which is itself a nucleic acid containing or presumed to contain a target nucleic acid sequence of interest. The term “sample” thus includes a sample of nucleic acid (genomic DNA, cDNA, RNA), cell, organism, tissue, fluid, or substance including but not limited to, for example, vaginal or anal swabs, amniotic fluid, whole blood, plasma, serum, spinal fluid, urine, stool, intestinal and genitourinary tracts, blood cells, samples of in vitro cell culture constituents, microbial specimens, and objects or specimens that have been “marked” with nucleic acid tracer molecules.

As used herein, “target nucleic acid sequence” refers to a region of a nucleic acid of interest and that is unique to GBS bacteria. The target nucleic acid sequence is the oligo- or poly-nucleotide sequence of the gene of interest that is selected for extension, replication, amplification and/or detection. In one embodiment, the gene of interest encodes a cell wall protein, and the “target nucleic acid sequence” encodes a region of the protein responsible for anchoring the protein to the cell wall. In one embodiment, the “target nucleic acid sequence” resides between two primer sequences used for amplification. In other cases the target may be a nucleic acid that is not amplified.

As used herein, “isolated” when used in reference to a polynucleotide (including an oligonucleotide) or a polypeptide means that a naturally occurring nucleotide or amino acid sequence has been removed from its normal cellular environment or is synthesized in a non-natural environment (e.g., artificially synthesized). Thus, an “isolated” polynucleotide (including an oligonucleotide) or an “isolated” polypeptide may be in a cell-free solution or placed in a different cellular environment. The term “isolated” does not imply that the nucleotide or amino acid sequence is the only polynucleotide or polypeptide present, but that it is essentially free (about 90-95%, up to 99-100% pure) of non-polynucleotide or non-polypeptide material naturally associated with it.

As used herein, an “oligonucleotide primer” and a “primer” are used interchangeably in their most general sense to include any length of nucleotides which, when used for amplification purposes, can provide a free 3′ hydroxyl group for the initiation of DNA synthesis by a DNA polymerase, either using a RNA or a DNA template. DNA synthesis results in the extension of the primer to produce a primer extension product complementary to the nucleic acid strand to which the primer has hybridized. Generally, the primer comprises from 8 to 100 nucleotides, preferably from 15 to 50 nucleotides and even more preferably from 15 to 35 nucleotides. The primers of the present invention may be synthetically produced by, for example, the stepwise addition of nucleotides or may be fragments, parts, portions or extension products of other nucleotide acid molecules.

As used herein, “probe” refers to a labeled oligonucleotide, which forms a duplex structure with a sequence in the target nucleic acid, due to complementarily of at least one sequence in the probe with a sequence in the target region. Such probes are useful for identification of a target nucleic acid sequence for GBS according to the invention, including the exemplified CAMP factor and gbs1539 genes of GBS. Generally, the probe comprises from 8 to 100 nucleotides, preferably from 15 to 50 nucleotides and even more preferably from 15 to 35 nucleotides.

“Complementary” refers to the broad concept of sequence complementarity between regions of two polynucleotide strands or between two nucleotides through base-pairing. It is known that an adenine nucleotide is capable of forming specific hydrogen bonds (“base pairing”) with a nucleotide which is thymine or uracil. Similarly, it is known that a cytosine nucleotide is capable of base pairing with a guanine nucleotide.

As used herein, the phrase “extension product” refers to the nucleic acid product of an extension reaction catalyzed by a template-dependent nucleic acid extending enzyme, e.g., by PCR. An “extension product” has been extended by at least one nucleotide by a template-dependent nucleic acid extending enzyme.

As used herein, “detecting the presence of an extension product” refers to determining the presence of an extension product in a sample or determining the amount of an extension product in a sample as an indication of the presence or amount of a target nucleic acid sequence in a sample. The amount (e.g., copy number) of a target nucleic acid sequence that can be measured or detected is preferably about 1 molecule to 10⁷ molecules, more preferably about 5 molecules to 10³ molecules and most preferably about 10 molecules to 10² molecules. Preferably there is a direct correlation between the amount of the target nucleic acid sequence and the signal generated by the detected nucleic acid.

As used herein, “amplifying” refers to the generation of additional copies of a nucleic acid sequence, i.e., the generation of extension products from primers. A variety of methods have been developed to amplify nucleic acid sequences, including the polymerase chain reaction (PCR). PCR amplification of a nucleic acid sequence generally results in the exponential amplification of a nucleic acid sequence(s) and or fragments thereof.

As used herein, the term “hybridizes” when used in reference to an oligonucleotide primer, refers to the formation of a hydrogen-bonded base paired duplex with a nucleic acid having a sequence sufficiently complementary to that of the oligonucleotide primer to permit the formation of such a duplex. As the term is used herein, exact complementarity between an oligonucleotide primer and a nucleic acid sequence is not required, with mismatches permitted as long as the resulting duplex is a substrate for extension by a template-dependent nucleic acid extending enzyme. A nucleic acid sequence is “sufficiently complementary” to an oligonucleotide primer if the primer can form a duplex with a molecule comprising the nucleic acid sequence at 55° C. that can be extended by at least one nucleotide by a template-dependent nucleic acid extending enzyme, e.g., a polymerase, in a solution comprising 10 mM Tris-HCl, pH 8.8, 50 mM KCl, 2.0 mM MgCl₂ and 200 μM each of dATP, dCTP, dGTP and dTTP. A “primer which hybridizes” to a polynucleotide sequence (e.g., SEQ ID NO:1) is complementary to the sequence or its complementary sequence thereof.

As used herein, the “CAMP (Christie-Atkins-Munch-Petersen) factor” is a diffusible extracellular protein and is produced by the majority of GBS. The gene encoding CAMP factor, the cfb gene (SEQ ID NO:2) (gi:840865), is present in virtually every GBS isolate.

As used herein, the terms “nucleic acid”, “polynucleotide” and “oligonucleotide” refer to primers, probes, and oligomer fragments to be detected, and shall be generic to polydeoxyribonucleotides (containing 2-deoxy-D-ribose), to polyribonucleotides (containing D ribose), and to any other type of polynucleotide which is an N-glycoside of a purine or pyrimidine base, or modified purine or pyrimidine bases. There is no intended distinction in length between the term “nucleic acid” and “polynucleotide” and “oligonucleotide”, and these terms will be used interchangeably. These terms refer only to the primary structure of the molecule. Thus, these terms include double- and single-stranded DNA, as well as double- and single-stranded RNA.

As used herein, the phrase “internal amplification control” refers to a double- or single-stranded nucleic acid molecule that is added to a nucleic acid amplification reaction to serve as a control for the activity of the template-dependent nucleic acid extending enzyme used in such reaction. An internal amplification control template useful according to the methods disclosed herein is amplified using the same primer pairs which are used to amplify the gbs1539 gene of GBS or a pair of primers which are used to amplify the CAMP factor gene. An example of internal amplification control of the present invention comprises a sequence of SEQ ID NO:9 or 12.

As used herein, the phrase “template-dependent nucleic acid extending enzyme” refers to an enzyme that catalyzes the template-dependent addition of nucleotides to the 3′ end of a nucleic acid strand hybridized to a substantially complementary template nucleic acid strand. Examples of such enzymes include, but are not limited to DNA polymerases.

As used herein, the term “aligning” when used in reference to nucleic acid sequences means arranging one or more sequences relative to another such that the greatest number of identical nucleotides are aligned with each other. BCM Search Launcher (via hypertext transfer protocol at //searchlauncher.bcm.tmc.edu/), formatted with BOXSHADE 3.2.1 on the Swiss EMBnet node server (available via hypertext transfer protocol on the world wide web at ch.embnet.org/software/BOX_form.html) can be used for primer sequence alignments. Multiple sequence alignments can also be performed using the BLAST suite of programs available from the NCBI website (see below).

The present invention features a rapid and accurate PCR-based assay for Streptococcus agalactiae, the organism responsible for neonatal Group B Streptococcal (GBS) infections. Standard molecular biology techniques known in the art and not specifically described herein may be found in a variety of standard laboratory manuals including: Sambrook et al., Molecular Cloning: A Laboratory Manual, Cold Springs Harbor Laboratory, New York (1992).

In one embodiment, the present invention identifies and utilizes specific primers and probes specific for the gbs1539 gene in GBS, which can be utilized in various PCR assays for specific and rapid identification of GBS in samples. The specific primers so identified can be used as a mixture to aid in increasing the sensitivity of screening for GBS using PCR. Moreover, the primers and probes identified herein can be used in real time PCR for rapid and convenient identification of GBS in clinical samples.

gbs1539 and Oligonucleotide Design:

The present invention provides compositions and methods for GBS detection utilizing a distinct gene gbs1539. The gbs1539 target is chosen from the sequence of the Streptococcus agalactiae genome (Glaser et al. (2002), Mol Microbiol, 45:1 499-1513; accession number gi:24413042, locus SAG76685, SEQ ID NO:1), serogroup III strain NEM316, since serotype III strains are responsible for 80% of neonatal GBS meningitis cases (Schuchat (1998), Clin Microbiol Rev, 11 :497-513; Nizet and Rubens (2000), In Gram-positive pathogens. Fischetti V A, Novick R P, Ferretti J J, Portnoy D A, and Rood J I (eds). Washington D.C.: American Society for Microbiology Press). As used herein, the term “gsb1539 gene” refers to a polynucleotide (either single stranded or double stranded) which comprises the sequence of SEQ ID NO:1 or its complementary sequence thereof. The term “gbs1539” also contemplate any corresponding variants present in various isolates of GBS.

| | |
|---|---|
| gbs 1539 CDS (gi:24413042, Genbank ID. AL766851 or AE014259) | SEQ ID NO: 1 |
| ATGAAAGTGAAAAATAAGATTTTAACGATGGTAGCACTTACTGTCTTAACATGTG | |
| CTACTTATTCATCAATCGGTTATGCTGATACAAGTGATAAGAATACTGACACGAG | |
| TGTCGTGACTACGACCTTATCTGAGGAGAAAAGATCAGATGAACTAGACCAGTCT | |
| AGTACTGGTTCTTCTTCTGAAAATGAATCGAGTTCATCAAGTGAACCAGAAACAA | |
| ATCCGTCAACTAATCCACCTACAACAGAACCATCGCAACCCTCACCTAGTGAAGA | |
| GAACAAGCCTGATGGTAGAACGAAGACAGAAATTGGCAATAATAAGGATATTTC | |
| TAGTGGAACAAAAGTATTAATTTCAGAAGATAGTATTAAGAATTTTAGTAAAGCA | |
| AGTAGTGATCAAGAAGAAGTGGATCGCGATGAATCATCATCTTCAAAAGCAAAT | |
| GATGGGAAAAAAGGCCACAGTAAGCCTAAAAAGGAACTTCCTAAAACAGGAGAT | |
| AGCCACTCAGATACTGTAATAGCATCTACGGGAGGGATTATTCTGTTATCATTAA | |
| GTTTTTACAATAAGAAAATGAAACTTTATTAA | |

gbs1539 is one of approximately 187 genes that are unique to S. agalactiae. gbs1539 is specifically chosen due to its lack of homology with any other sequence in the public databases (Glaser, et al. (2002), supra). The coding sequence of gbs1539 is 579 base pairs (see SEQ ID NO:1), encoding a putative protein of 192 amino acids. The G-C content of the gbs1539 gene is 36% compared to 33% for the CAMP-factor gene. While the function of the gbs1539 protein product is unknown, the protein contains an LPXTG motif (Navarre and Schneewind (1999), Microbiol Mol Biol Rev, 63:174-229) required for anchoring of proteins to the cell wall of gram-positive bacteria. As one of only 30 open reading frames in the GBS genome containing a cell wall sorting signal (Glaser, et al. (2002), supra), the gbs1539 protein may be important for GBS viability or pathogenesis. Hence, the gene is expected to be conserved among clinical isolates, a feature which can be used to advantage for the development of a PCR based assay for GBS contamination as described herein.

The present invention, therefore, is directed to the use of novel compositions and methods for GBS detection utilizing gbs1539.

In one embodiment, a region within nucleotide positions 50 to 400 of gbs1539 is detected.

In another embodiment, a region within nucleotide positions 100 to 350 of gbs1539 is detected.

In another embodiment, a region within nucleotide positions 106 to 305 of gbs1539 is detected.

Furthermore, the present invention identifies and utilizes specific primers and probes specific for the gbs1539 gene in GBS, which can be utilized in various PCR assays for specific and rapid identification of GBS in samples. The specific primers so identified can be used as a mixture to aid in increasing the sensitivity of screening for GBS using PCR. Moreover, the primers and probes identified herein can be used in real time PCR for rapid and I convenient identification of GBS in clinical samples.

General criteria for primer or probe designing is followed when designing primers or probes useful to the present invention.

Primers and probes useful according to the invention are also designed to have a particular melting temperature (Tm) by the method of melting temperature estimation. For example, commercial programs, including Oligo™ Primer Design and programs available on the internet, including Primers and Oligo Calculator can be used to calculate a Tm of a nucleic acid sequence useful according to the invention. Preferably, the Tm of an amplification primer useful according to the invention, as calculated for example by Oligo Calculator, is preferably between about 50 and 65° C. and more preferably between about 55 and 65° C. Preferably, the Tm of a probe useful according to the invention is at least 3° C. (e.g., 4° C., 5° C. or 6° C.) higher than the Tm of the corresponding amplification primers.

Typically, selective hybridization occurs when two nucleic acid sequences are substantially complementary (at least about 65% complementary over a stretch of at least 14 to 25 nucleotides, preferably at least about 75%, more preferably at least about 90% complementary). See Kanehisa, M., 1984, Nucleic Acids Res. 12: 203, incorporated herein by reference. As a result, it is expected that a certain degree of mismatch at the priming site is tolerated. Such mismatch may be small, such as a mono-, di- or tri-nucleotide. Alternatively, a region of mismatch may encompass loops, which are defined as regions in which there exists a mismatch in an uninterrupted series of four or more nucleotides.

Numerous factors influence the efficiency and selectivity of hybridization of the primer or probe to a second nucleic acid molecule. These factors, which include primer/probe length, nucleotide sequence and/or composition, hybridization temperature, buffer composition and potential for steric hindrance in the region to which the primer is required to hybridize, will be considered when designing oligonucleotide primers according to the invention.

A positive correlation exists between primer/probe length and both the efficiency and accuracy with which a primer/probe will hybridize to a target sequence. In particular, longer sequences have a higher melting temperature (TM) than do shorter ones, and are less likely to be repeated within a given target sequence, thereby minimizing promiscuous hybridization. Primer/probe sequences with a high G-C content or that comprising palindromic sequences tend to self-hybridize, as do their intended target sites, since unimolecular, rather than bimolecular, hybridization kinetics are generally favored in solution. Hybridization temperature varies inversely with primer/probe hybridization efficiency, as does the concentration of organic solvents, e.g. formamide, that might be included in a priming reaction or hybridization mixture, while increases in salt concentration facilitate binding. Under stringent annealing conditions, longer hybridization probes, or synthesis primers, hybridize more efficiently than do shorter ones, which are sufficient under more permissive conditions. Stringent hybridization conditions typically include salt concentrations of less than about 1M, more usually less than about 500 mM and preferably less than about 200 mM. Hybridization temperatures range from as low as 0° C. to greater than 22° C., greater than about 30° C., most often in excess of about 37° C.

Longer fragments may require higher hybridization temperatures for specific hybridization. As several factors affect the stringency of hybridization, the combination of parameters is more important than the absolute measure of a single factor. Oligonucleotide primers and probes can be designed with these considerations in mind and synthesized according to the following methods.

In one embodiment, the design of a particular oligonucleotide primer for the purpose of sequencing, PCR, or for use in identifying target nucleic acid molecules of GBS involves selecting a sequence that is capable of recognizing the target sequence, but has a minimal predicted secondary structure. The oligonucleotide sequence binds only to a single site in the target nucleic acid sequence. Furthermore, the Tm of the oligonucleotide is optimized by analysis of the length and GC content of the Oligonucleotide. Furthermore, when designing a PCR primer useful for the amplification of genomic DNA, the selected primer sequence does not demonstrate significant matches to sequences in the GenBank database (or other available databases).

A useful primer for producing an extension product or a probe for the purpose of detecting the presence of GBS is selected to hybridize with the gsb1539 gene (e.g., SEQ ID NO:1 or its complementary sequence thereof). Preferably, a primer is selected such that it is perfectly complementary in its three 3′-terminal nucleotides to the target nucleic acid sequence (e.g., SEQ ID NO:1 or its complementary sequence thereof). The primer or probe may have one or more mismatches. Primers and probes according to the invention are preferably 8-100 nucleotides in length, preferably from 15 to 50 nucleotides and even more preferably from 15 to 35 nucleotides.

The specific design of one or more of the necessary primers to avoid extension of primers cross-hybridized to contaminating nucleic acid template from the source of the sample (e.g., human) can reduce or eliminate false positive results. A potential primer or probe sequence may be aligned against sequences in public databases so as to ensure the least homology against any sequences in the databases, e.g., against nucleotide sequence database from which the sample is derived (e.g., human sequence databases). Such sequence alignment can be performed by one of skill in the art manually, i.e., by eye, or, preferably, the alignment can be performed by computer using software that is widely available. For example, where a homologous sequence is already known, the “Blast 2 Sequences” program (b12seq; Tatusova & Madden (1999), FEMS Microbiol. Lett. 174:247-250) can be used. The program is available through the NCBI website and can be used with default alignment parameters. This program produces the alignment of two given sequences using the BLAST engine for local alignment. Default parameters (for use with the BLASTN program only) are as follows: Reward for a match: 1; Penalty for a mismatch: −2; Strand option Both strands; open gap penalty 5; extension gap penalty 2; gap x_dropoff 50; expect 10.0; word size 11; and Filter (checked).

Where homologs are not known, or where one, for example, wishes to determine whether there are homologs with a higher degree of homology than a known homolog, BLAST alignment can be performed against nucleic acid sequences from the recombinant host species. For example, the genome sequence of the recombinant host can be searched and similar sequences aligned. For this purpose, a BLAST alignment can be preformed using the BLASTN program of the Basic BLAST suite of programs (Basic BLAST, Version 2.0, Altschul et al., 1997, Nucleic Acids Res. 25: 3389-3402) set with default parameters (descriptions=500; alignments=100; expect=10; filter=off; matrix=BLOSUM62).

Examples of gbs1539 primers identified for the present invention include, but are not limited to sequences shown below and their complementary sequences thereof:

SEQ ID NO: 3: gbs1539-F
ACGAGTGTCGTGACTACGACCTTA
SEQ ID NO: 4: gbs1539-R
TCTGTCTTCGTTCTACCATCAGGC

Example of gbs1539 probe includes, but is not limited to:

SEQ ID NO: 5: gbs1539-P
6-FAM/ACCTACAACAGAACCATCGCAACCCT/BHQ-1

Examples of CAMP primers and probes include, but are not limited to SEQ ID NOs. 6-8 as described herein below.

The primer or probe of the present invention, as described herein above and below, may be labeled with a detectable label. A wide variety of labels and conjugation techniques are known by those skilled in the art and may be used in various nucleic acid and amino acid assays. Means for producing labeled primers or probes for detecting sequences related to polynucleotides include oligolabeling, nick translation, end-labeling or PCR amplification using a labeled nucleotide. Suitable reporter molecules or labels, which may be used include radioisotopes or radiolabeled molecules, fluorescent molecules, fluorescent antibodies, enzymes, or chemiluminescent catalysts. Within certain embodiments of the invention, the probe may contain one or more labels such as a fluorescent or enzymatic label (e.g., quenched fluorescent pairs, or, a fluorescent label and an enzyme label), or a label and a binding molecule such as biotin (e.g., the probe, either in its cleaved or uncleaved state, may be covalently or non-covalently bound to both a label and a binding molecule (see also, e.g., U.S. Pat. No. 5,731,146, incorporated by reference in its entirety).

The probes of the present invention may also be linked to a solid support either directly, or through a chemical linker. Representative examples of solid supports include silicaceous, cellulosic, polymer-based, or plastic materials.

The methods of the invention presented herein include an isolated oligonucleotide comprising a sequence selected from the group consisting of SEQ ID NOs. 3-5 and their complementary sequences thereof.

The present invention provides a pair of primers, with a first primer comprising the sequence of SEQ ID NO:3 or its complementary sequence thereof, and a second primer comprising a sequence of SEQ ID NO:4 or its complementary sequence thereof. The primers hybridize to a sequence comprising SEQ ID NO:1 or its complementary sequence thereof, and produce extension products that are copies of a portion of SEQ ID NO:1.

The present invention also provides for a specific probe (SEQ ID NO:5) designed to recognize the extension product produced by the primers, e.g., from SEQ ID NO:1, allowing real-time detection by using fluorescence measurements.

Primers and probes, as described herein above and below, may be synthesized or obtained and/or prepared directly from a target cell or organism utilizing standard techniques (see, e.g., Sambrook et al., “Molecular Cloning: A Laboratory Manual”, Cold Spring Harbor, 1989), or prepared utilizing any of a wide variety of a techniques, including for example, PCR, NASBA reverse transcription of RNA, SDA branched-chain DNA and the like. Methods for preparing oligonucleotides of specific sequence are known in the art, and also include, for example, cloning and restriction digest analysis of appropriate sequences and direct chemical synthesis. Once designed, oligonucleotides are prepared by a suitable chemical synthesis method, including, for example, the phosphotriester method described by Narang et al., 1979, Methods in Enzymology, 68:90, the phosphodiester method disclosed by Brown et al., 1979, Methods in Enzymology, 68:109, the diethylphosphoramidate method disclosed in Beaucage et al., 1981, Tetrahedron Letters, 22:1859, and the solid support method disclosed in U.S. Pat. No. 4,458,066, or by other chemical methods using either a commercial automated oligonucleotide synthesizer (which is commercially available) or VLSIPS technology.

It is well known by those with skill in the art that oligonucleotides can be synthesized with certain chemical and/or capture moieties, such that they can be coupled to solid supports. Suitable capture moieties include, but are not limited to, biotin, a hapten, a protein, a nucleotide sequence, or a chemically reactive moiety. Such oligonucleotides may either be used first in solution, and then captured onto a solid support, or first attached to a solid support and then used in a detection reaction. An example of the latter would be to couple a downstream probe molecule to a solid support, such that the 5′ end of the downstream probe molecule comprised a fluorescent quencher.

The primers and probes of the present invention need not be perfectly complementary, and indeed, may be purposely different by one, two, three or more nucleotides from the target nucleic acid sequence.

PCR Analysis:

The selected primer sequence is then used to produce an extension product. Preferably, the extension produce is produced by PCR amplification. The presence of an extension product of an expected size detected after gel electrophoresis of PCR extension products confirms the presence of GBS in the sample. It is further contemplated that detection methods of the present invention can use other types of enzyme-mediated amplification, for example 3SR (Self-Sustained Sequence Replication; Gingeras et al. (1990), Annales de Biologie Clinique, 48(7): 498-501; Guatelli et al. (1990), Proc. Natl. Acad. Sci. U.S.A., 87: 1874), or SDA (Strand Displacement Amplification; Walker (1994), Nucleic Acids Res. 22:2670-7).

PCR-based detection assays rely upon the ability of a set of primers specific for a given nucleic acid sequence to direct the amplification of a target sequence from among a background of non-target sequences. PCR amplification of the present invention takes advantage of the unique GBS gene gbs1539 and produces an extension product using gbs1539 as template.

The present invention provides a method of detecting the presence of a GBS in a sample, comprising (a) contacting the sample with a primer which hybridizes to the sequence of SEQ ID NO:1 to produce an extension product from the primer and detecting the presence of the extension product from the primer.

In one embodiment, the primer is labeled with a detectable label, as discussed above.

In another preferred embodiment, the subject method comprises contacting the sample with a pair of primers, with a first primer comprises a sequence of SEQ ID NO:3 or its complementary sequence thereof and a second primer comprises a sequence of SEQ ID NO:4 or its complementary sequence thereof.

In a preferred embodiment, the method comprising contacting the sample with the pair of primers as described above in the presence of a probe comprising the sequence of SEQ ID NO:5 or its complementary sequence thereof.

In a more preferred embodiment, the method comprising contacting the sample with the pair of primers as described above in the presence of a probe comprising the sequence of SEQ ID NO:5 or its complementary sequence thereof, and further in the presence of an internal amplification control template.

The probe used in the present invention, e.g., a probe comprising SEQ ID NO:5, may be double labeled with a fluorophore at one end and a quencher at the other end, so when the probe is intact (i.e., not hybridized to the extension product) the flourophore does not emit a detectable fluorescent signal.

In one embodiment, the probe of the present invention (e.g., a probe comprising SEQ ID NO:5) is labeled with a fluorophore the 5′ and a quencher at the 3′ end.

In another embodiment, the probe of the present invention (e.g., a probe comprising SEQ ID NO:5) is labeled with a fluorophore the 3′ and a quencher at the 5′ end.

In another embodiment, the probe of the present invention (e.g., a probe comprising SEQ ID NO:5) contains an internal quencher moiety.

A number of template dependent processes are available to amplify the target sequences of interest present in a sample for the present invention. One of the best known amplification methods is the polymerase chain reaction (PCR) which is described in detail in U.S. Pat. Nos. 4,683,195, 4,683,202 and 4,800,159, each of which is incorporated herein by reference in its entirety. Briefly, two primer sequences are prepared which are complementary to regions on opposite complementary strands of the target sequence. An excess of deoxynucleoside triphosphates is added to a reaction mixture along with a DNA polymerase (e.g., Taq polymerase). If the target sequence is present in a sample, the primers will bind to the target and the polymerase will cause the primers to be extended along the target sequence by adding on nucleotides. By raising and lowering the temperature of the reaction mixture, the extended primers will dissociate from the target to form reaction products, excess primers will bind to the target and to the reaction product and the process is repeated. Preferably reverse transcription and PCR amplification procedure may be performed in order to quantify the amount of mRNA amplified. Polymerase chain reaction methodologies are well known in the art.

Another useful method for amplification is the ligase chain reaction (referred to as LCR), disclosed in Eur. Pat. Appl. Publ. No. 320,308 (specifically incorporated herein by reference in its entirety). In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR bound ligated units dissociate from the target and then serve as “target sequences” for ligation of excess probe pairs. U.S. Pat. No. 4,883,750, incorporated herein by reference in its entirety, describes an alternative method of amplification similar to LCR for binding probe pairs to a target sequence.

Q beta Replicase, described in PCT Intl. Pat. Appl. Publ. No. PCT/US87/00880, incorporated herein by reference in its entirety, may also be used as still another amplification method in the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′-[oc-thio]triphosphates in one strand of a restriction site (Walker et al., 1992, incorporated herein by reference in its entirety), may also be useful in the amplification of nucleic acids in the present invention.

Strand Displacement Amplification (SDA) is another method of calcifying out isothermal amplification of nucleic acids which involves multiple rounds of strand displacement and synthesis, i.e. nick translation. A similar method, called Repair Chain Reaction (RCR) is another method of amplification which may be useful in the present invention and is involves annealing several probes throughout a region targeted for amplification, followed by a repair reaction in which only two of the four bases are present. The other two bases can be added as biotinylated derivatives for easy detection. A similar approach is used in SDA.

Numerous different PCR or QPCR protocols are known in the art and exemplified herein below and can be directly applied or adapted for use in the presently-described methods. The subject PCR or QPCR may be performed on any suitable instrument, include, but are not limited to the automated PCR instruments sold by Stratagene under the tradenames, Mx4000 and Mx3000; to PCR instruments sold by “X” under the trademanes ABI7700, ABI7000 (ABI), MJ Opticon (MJ research); and BioRad iCycler (Biorad).

Quantitative PCR (QPCR) (also referred as real-time PCR) is preferred under some circumstances because it provides not only a quantitative measurement, but also reduced time and contamination. As used herein, “quantitative PCR (or a real time QPCR)” refers to the direct monitoring of the progress of a PCR amplification as it is occurring without the need for repeated sampling of the reaction products. In quantitative PCR, the reaction products may be monitored as they are generated and are tracked after they rise above background but before the reaction reaches a plateau. The number of cycles required to achieve a chosen level of fluorescence varies directly with the concentration of amplifiable targets at the beginning of the PCR process, enabling a measure of signal intensity to provide a measure of the amount of target DNA in a sample in real time.

In a preferred embodiment, a labeled probe is used to detect the extension product generated by PCR amplification. Any detection probe utilizing a labeled probe may be used, e.g., such as Taqman or molecular beacon detection known in the art.

In a preferred embodiment, the PCR is a hydrolytic quantitative PCR assay that utilizes two novel proteins for amplification of target DNA and hydrolysis of hybridized probes (e.g., as described in U.S. Pat. Nos. 6,548,250 and 6,528,254, the entirety of each is hereby incorporated by reference). This PCR approach utilizes an exonuclease-deficient Pfu polymerase for amplification, and a flap endonuclease for probe cleavage. As Pfu polymerase extends the primers, it encounters hybridized probe, thereby displacing the 5′ end of the probe from the template. Flap endonuclease recognizes the structure of the displaced probe-template junction, clips the 5′ end of the probe at the internal phosphodiester bond, and releases the probe's fluorophore from probe's quencher. A signal is generated upon probe cleavage that is proportional to the amount of PCR product in the reaction. The intensity of the fluorescence increases as a function of the synthesis of additional amplicons during the course of subsequent cycles of PCR.

Variations on the exact amounts of the various reagents and on the conditions for the PCR (e.g., buffer conditions, cycling times, etc.) that lead to similar amplification or detection results are known to those of skill in the art and are considered to be equivalents. In one embodiment, the subject QPCR detection has a sensitivity of detecting less than 50 copies (preferably less than 25 copies, more preferably less than 15 copies, still more preferably less than 10 copies) of gbs1539 DNA (e.g., genomic or cDNA) in a sample. In one embodiment, a hot-start PCR reaction is performed (e.g., using a hot start Taq DNA polymerase such as SureStart Taq DNA polymerase from Stratagene) so as to improve PCR reaction by decreasing background from non-specific amplification and to increase amplification of the desired extension product.

The PCR or QPCR reaction of the present invention may contain various controls. Such controls should include a “no template” negative control, in which primers, buffer, enzyme(s) and other necessary reagents (e.g., MgCl₂, nucleotides) are cycled in the absence of added test sample. A positive control including a known target template should also be run in parallel.

Both positive control and negative control may be included in the amplification reaction. A single reaction may contain either a positive control, a negative control, or a sample template, or a single reaction may contain both a sample template and a positive control.

In one embodiment, the gbs1539 positive internal control is a cloned gene fragment that is flanked by the GBS primer-binding sites. Therefore, the internal control DNA will be amplified in PCR by the GBS primers, but the internal sequence of the amplicon will be different from the GBS target (e.g., a fragment of the plastocyanin (PC) gene from Arabidopsis thaliana). A distinct fluorogenic probe that binds only to the internal control sequence will be used to detect amplification of the internal control. If the internal control sequence is detected, then PCR was successful. If the internal control amplification failed, this indicates the presence of PCR inhibitors in the clinical sample.

In a preferred embodiment, the gbs1539 positive internal control is shown as follows:

| | |
|---|---|
| CTCGAG [symbol] GGATGGTGACATGACAAACGGCAA | (SEQ ID NO: 14) |
| GGCTTTTTAGGTGATTTATTTAAAACGGCAACTCGTTTTGTTGTAGGTCGTTTCTC | |
| TCTTTTAAGATTGGACGCCTCGCCGTTTCTTTTATTTTCTAGCTTATTGTAGTTGTC | |
| CATGCTTGCTTAA [symbol] GGATCC | |

Cloning sites (XhoI/BamHI) are underlined, Gbs1539 primer binding sites are bolded/italicized, internal control probe binding sequence is bolded/underlined. An internal control probe that specifically binds to the gbs1539 internal control is provided. In one embodiment, the same internal control probe used for CAMP internal control (SEQ ID NO:13) is also used for the detection of gbs1539 internal control.

Alternative and/or Additional positive controls may be used in the PCR reaction for gauging inhibitory activity. Clinical samples contain many substances capable of inhibiting PCR, including but not limited to feces, cellular debris, heme, or urea. The sample may be spiked with a control nucleic acid template. By designing distinct nucleic acid species that contain primer binding sites for GBS target, amplification of spiked nucleic acid can be monitored independently of genomic DNA target. If amplification of such internal control sequence fails, negative results for target gene cannot be confirmed, as inhibition of PCR may have occurred.

In one embodiment, the sample is spiked with a nucleic acid (e.g., a vector or PCR product) containing a CAMP factor sequence. For this CAMP-factor target, oligonucleotides are designed that contain the exact binding sequences for CAMP-factor primers at the 5′ end, but also contain primer-binding sites at their 3′ ends for a fragment of the plastocyanin (PC) gene of Arabidosis thailana (e.g., SEQ ID NOs. 9-11). The PC gene is arbitrarily chosen due to its distant evolutionary relationship to GBS, thereby making it unlikely that nonspecific hybridizations or side-reactions could occur during cycling. The PC fragment containing the CAMP-factor primer binding sites and appropriate 5′-cloning sites is amplified by PCR (e.g., SEQ ID NO:12), digested with the corresponding restriction enzymes, and cloned into pBluescript. The plasmid containing the PC insert is directly utilized as an internal control sequence in the CAMP-factor PCR.

In another embodiment, an internal control probe is designed (e.g., SEQ ID NO:13) to anneal specifically to the PC fragment contained within the internal control plasmid. The probe is labeled with HEX to distinguish its signal from the target (FAM).

The CAMP-factor cds is shown as

| | |
|---|---|
| ATGAACGTTACACATATGATGTATCTATCTGGAACTCTAGTGGCTGGTGCA: | SEQ ID NO: 2 |
| TTGTTATTTTCACCAGCTGTATTAGAAGTACATGCTGATCAAGTGACAACTCCACA | |
| AGTGGTAAATCATGTAAATAGTAATAATCAAGCCCAGCAAATGGCTCAAAAGCTT | |
| GATCAAGATAGCATTCAGTTGAGAAATATCAAAGATAATGTTCAGGGAACAGAT | |
| TATGAAAAACCGGTTAATGAGGCTATTACTAGCGTGGAAAAATTAAAGACTTCAT | |
| TGCGTGCCAACCCTGAGACAGTTTATGATTTGAATTCTATTGGTAGTCGTGTAGA | |
| AGCCTTAACAGATGTGATTGAAGCAATCACTTTTTCAACTCAACATTTAACAAAT | |
| AAGGTTAGTCAAGCAAATATTGATATGGGATTTGGGATAACTAAGCTAGTTATTC | |
| GCATTTTAGATCCATTTGCTTCAGTTGATTCAATTAAAGCTCAAGTTAACGATGTA | |
| AAGGCATTAGAACAAAAAGTTTTAACTTATCCTGATTTAAAACCAACTGATAGAG | |
| CTACCATCTATACAAAATCAAAACTTGATAAGGAAATCTGGAATACACGCTTTAC | |
| TAGAGATAAAAAAGTACTTAACGTCAAAGAATTTAAAGTTTACAATACTTTAAAT | |
| AAAGCAATCACACATGCTGTTGGAGTTCAGTTGAATCCAAATGTTACGGTACAAC | |
| AAGTTGATCAAGAGATTGTAACATTACAAGCAGCACTTCAAACAGCATTAAAATA | |
| A | |

Positive control primers and probes for producing an extension product from CAMP-factor include, but are not limited to:

SEQ ID NO: 6 GBSf2 (CAMP-factor forward primer)
TGAGGCTATTACTAGCGTGGAAAAA
SEQ ID NO: 7 GBSr2 (CAMP-factor reverse primer)
CATCTGTTAAGGCTTCTACACGACTA
SEQ ID NO: 8 GBSp2 (CAMP-factor probe)
6-FAM/ACTTCATTGCGTGCCAACCCTGAGACAGT/BHQ-1
SEQ ID NO: 9: Arabidopsis. thaliana plastocyanin fragment
(for internal control), from gi: 166789
GGATGGTGACATGACAAACGGCAAGGCTTTTTAGGTGATTTATTTAAAACGGCAA
CTCGTTTTGTTGTAGGTCGTTTCTCTCTTTTAAGATTGGACGCCTCGCCGTTTCTTT
TATTTTCTAGCTTATTGTAGTTGTCCATGCTTGCTTAA
SEQ ID NO: 10: AthPC-F (Internal control forward primer for
CAMP-factor target)
TATACTCGAGTGAGGCTATTACTAGCGTGGAAAAAGGATGGTGACATGACAAAC
GGC
SEQ ID NO: 11: AthPC-R (Internal control reverse primer for
CAMP-factor target)
TATAGGATCCCATCTGTTAAGGCTTCTACACGACTATTAAGCAAGCATGGACAAC
TACAATAAGC
SEQ ID NO: 12: Cloned internal control sequence for CAMP-factor
target, between Xhol and BamHl (underlined) restriction sites
CTCGAGTGAGGCTATTACTAGCGTGGAAAAAGGATGGTGACATGACAAACGGCA
AGGCTTTTTAGGTGATTTATTTAAAACGGCAACTCGTTTTGTTGTAGGTCGTTTCT
CTCTTTTAAGATTGGACGCCTCGCCGTTTCTTTTATTTTCTAGCTTATTGTAGTTGT
CCATGCTTGCTTAATAGTCGTGTAGAAGCCTTAACAGATGGGATCC
SEQ ID NO: 13: PC-ICP (Internal control probe)
HEX/TGGTGACATGACAAACGGCAAGGCTT/BHQ-1

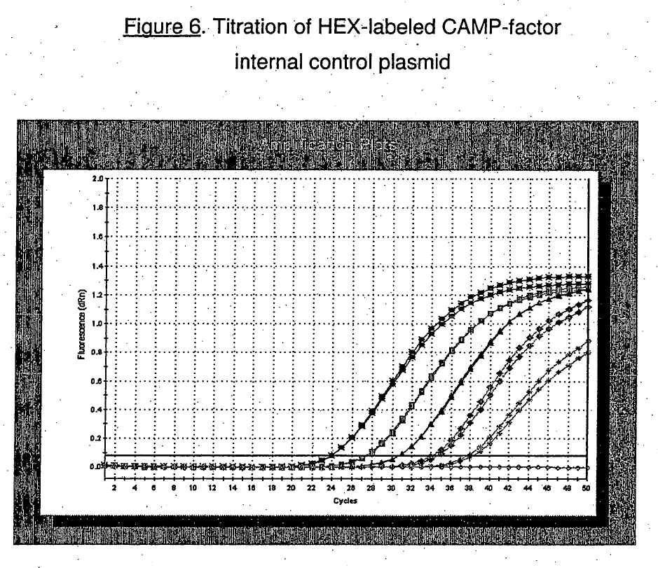

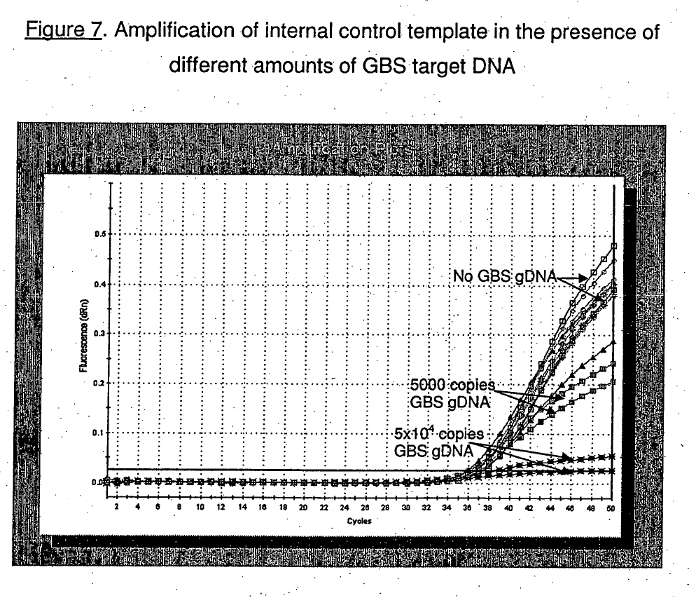

In one embodiment, using the CAMP-factor primers and HEX-labeled IC probe, the IC template was detected down to 50 copies in a concentration-dependent manner (FIG. 6). In FIG. 6, the assay contained the standard concentration of CAMP-factor primers/probe, and 200 nM HEX labeled internal control probe. The no-template control (orange line, open circles) contained TE in place of internal control plasmid. When plotting Ct vs. the log of the internal control concentration (standard curve, data not shown), the R² value was 0.998. (Note: for the no-template control, one of two replicates showed a small false-positive signal (equivalent to <5 copies), and was omitted from analysis shown here). When 100 copies of IC template was included in reactions containing a wide range of GBS genomic DNA concentrations, IC was efficiently amplified except when GBS genomic DNA exceeded 10⁴ copies (FIG. 7). In FIG. 7, the HEX channel signal is shown here. From 0 to 500 copies of GBS genomic DNA, the internal control signal shows nearly identical Ct and total fluorescence. The HEX fluorescence signal begins to weaken at 5000 copies and is barely detectable at 5×10⁴ copies of competing GBS genomic DNA. Such inhibition is expected in the presence of high amounts of target DNA, and does not preclude the use of the IC template to gauge inhibition. In experiments where inhibitors were purposefully spiked into GBS samples, the internal control failed to amplify, demonstrating the usefulness of the IC in identifying potential false-negative results.

In one embodiment, the same HEX-labeled PC probe (e.g., SEQ ID NO:13) will be used to detect the gbs1539 internal control template.

When fluorescence signal from a PCR reaction is monitored in real-time, the results can be displayed as an amplification plot, which reflects the change in fluorescence during cycling. This information can be used to derive the threshold cycle (Ct), from which the initial copy number may be quantified, e.g., as described in Higuchi et al. (1993, Biotechnology (NY) 11(9):1026-30, the entirety is hereby incorporated by reference). Here, Ct is defined as the cycle at which fluorescence is statistically significant above background. The threshold cycle is inversely proportional to the log of the initial copy number (e.g., Higuchi et al., supra). The more template that is initially present, the fewer the number of cycles required for the fluorescence signal to become detectable above background.

In one embodiment, the gbs1539 internal control (e.g., SEQ ID NO:13) and the gbs1539 primers (e.g., SEQ ID Nos. 3-4) are used, together with the internal control probe (e.g., SEQ ID NO:13), to detect GBS in a sample. The reaction may also contains ROX reference dye. The result is interpreted as follows:

If both FAM and HEX signals are detected, the sample is confirmed positive for GBS DNA and the test result is reported as “positive.” If only FAM signal is detected, but HEX signal is negative, the sample is confirmed positive for GBS DNA (excess target DNA out-competed the internal control template) and the test result is reported as “strong positive.” If FAM signal is negative, but HEX signal is positive, the sample, is confirmed negative for GBS DNA and the result is reported as “negative.” If both FAM and HEX signals are undetected, then there may be reagent failure or inhibitors present in the sample, the assay needs to be repeated with fresh reagents or the sample has to be treated to remove inhibitors before amplification.

Sample Preparation

The compositions and methods provided herein may be utilized to detect the presence of a desired target nucleic acid molecule, i.e., gbs1539 for GBS detection within a biological sample. Representative examples of biological samples include cultured (e.g., samples grown in a bacteriological medium) or clinical samples, including, but not limited to, samples from vaginal or anal swabs, whole blood, serum, plasma, urine, stool, and abscess or spinal fluids. Methods for generating target nucleic acid molecules may be readily accomplished by one of ordinary skill in the art given the disclosure provided herein and general knowledge of such procedures (see generally, Sambrook et al., Molecular Cloning: A Laboratory Manual (2d ed.), Cold Spring Harbor Laboratory Press, 1989).

In a particular aspect of the invention, a sample may be a bodily fluid derived from a pregnant female. Such a sample may be isolated prior to or at the time of delivery.

For example, clinical isolates of GBS can be obtained from individual patient swabs. In a preferred procedure, a sterile swab is used to obtain vaginal fluid samples. For example, Dacron or Rayon swabs with pre-scored handles can be used for sample collection. The swab is then placed in a sample collection tube.

In a protocol for immediate sample preparation, the swab/sample is transported immediately at room temperature for processing and analysis. If the swab/sample cannot be immediately processed, the swab/sample may be held at 0° C. to 8° C. for four hours or at room temperature for one hour. Longer storage time may be suitable too.

In one embodiment, for processing, lysis solution is added to the swab/sample, which is then swirled or agitated in the lysis solution for about 10-15 seconds. The tube containing the swab and lysis solution is heated at 85° C. for 5 minutes, and hybridization solution is mixed with the sample. At this point, samples may be stored for up to 24 hours at room temperature or for longer time at lower temperature.

In one embodiment, the swab contents are expressed by twirling the swab against the side of the tube, and the solution remaining in the tube may then be processed on an automated instrument. Generally, the solution remaining in the tube is filtered prior to further processing.

In another embodiment, the isolate is inoculated into selective media (TransVag broth), and grown overnight. The organisms can be subcultured onto blood agar plates and grown overnight to produce isolated colonies. Single colonies from each sample are streaked onto fresh agar plates to produce homogeneous cultures for each of the 82 original patients. Plates are transported to BioCrest, and stored at −20° C. until use.

In one embodiment, each isolate is initially tested in PCR with the gbs1539 primer/probe set. A single colony is transferred to 250 μl of Lysis Buffer (20 mM Tris, pH 7.4, 2 mM EDTA, 1.2% Triton X-100) and heated to 95° C. for 5 minutes. The sample is vortexed for ˜15 seconds, and 5 μl is directly added to the PCR for testing. Isolates testing negative in the first PCR are retested with both the gbs1539 and CAMP-factor primer/probe sets.

In another embodiment, the Lysis Buffer described above also contains 5 mg/ml Proteinase K and 30 mM DTT. The swab is placed into 500 μl of Lysis Buffer and briefly vortexed. The sample is heated at 70° C. for 8 minutes, and further heated to 95° C. for 2 minutes. The sample is then vortexed for about 15 seconds. 5 μl of sample is added directly to the PCR. A silica gel (e.g., StrataClean Resin, Cat. No. 400714, Stratagene) may be used to reduce the concentration of components in the sample that are inhibitory to PCR amplification (e.g., fetal calf serum, metabolic products, cell debris, etc.).

As discussed above, clinical samples may contain inhibitors for PCR reaction. In addition to identifying inhibited PCR reactions, an appropriate sample preparation method may be required to optimize genomic DNA release from bacteria and to reduce the number of inhibited samples.

In one embodiment, the IDI-Strep B kit utilizes a proprietary glass bead lysis method (IDI DNA extraction kit) to quickly release DNA from the gram-positive bacteria while simultaneously removing inhibitors (IDI Strep B assay manual, Ke et al., 2000, Development of conventional and real-time PCR assays for the rapid detection of group B Streptococcus. Clin Chem 46:324-331).

In another embodiment, simple resuspension of GBS liquid cultures or colonies from clinical isolates in a Tris-EDTA lysis buffer containing 1.2% Triton, followed by heating at 95° C. for 5 minutes and brief vortexing, lysate is efficiently amplified in the real-time PCR assays described herein above.

In another embodiment, proteinase K and DTT are included in the lysis buffer.

Other methods may be used to improve yield of DNA from gram-positive bacteria due to the difficulty in rupturing the cell wall. These include enzymatic digestions of the peptidoglycan cell wall (e.g. lysozyme or mutanolysin), chemical treatments, or mechanical methods (e.g. sonication, glass beads).

Methods to remove inhibitors that may be present in the lysate include, but are not limited to, the use of silica-based spin columns. Briefly, the DNA from the lysate is bound to the silica matrix in the presence of chaotropic salt, inhibitors are washed away, and the DNA is eluted into TE or sterile water. The methods and considerations necessary for PCR amplification are well known to those of skill in the art. Exemplary conditions are as provided in Examples.

Kits

On one aspect, a kit is provided containing reagents and instructions necessary to perform the GBS detection methods described herein. In a specific aspect, then, the kit can comprise an isolated primer and/or probe as described above herein.

In additional aspects, the kit can further comprise an internal control template, a positive control for gbs1539, a template-dependent nucleic acid extending enzyme (preferably a thermostable template-dependent nucleic acid extending enzyme, e.g., a Pfu DNA polymerase), a necessary buffer, additional reagents needed by the enzyme, such as MgCl₂, dNTPs, dUTP and/or a UDG enzyme.

### EXAMPLES

The following examples are put forth so as to provide those of ordinary skill in the art with a complete disclosure and description of how to make and use the methods and compositions of the invention, and are not intended to limit the scope of what the inventors regard as their invention.

### Example 1

### Materials and Methods

PCR Primers and Genomic DNA Preparation

PCR primers and probes were synthesized by IDT (Coralville, Iowa). Dye and quencher modifications of probes are indicated as described for SEQ ID NOs. 5, 8 and 13. All primers and probes were desalted and purified with either PAGE or HPLC purification, with the exception of the gbs1539 primers, which were only desalted. In some experiments, the gbs1539 primers were further PAGE purified for use in PCR.

GBS genomic DNA was prepared from overnight cultures of Streptococcus agalactiae cells (ATCC #BAA-61 1, 2603 V/R sequencing strain) using the DNeasy Tissue Kit (Qiagen, Valencia, Calif.) according to the manual's standard procedure for gram-positive bacteria. Copy number was estimated by DNA quantification on a Beckman spectrophotometer and conversion based upon the molecular weight of the S. agalactiae genome (2200 kb).

Non-GBS genomic DNAs were purchased from ATCC: Enterococcus faecalis (#7008021)), Lactococcus lactis (#19435D), Listeria monocytogenes (#1911 SD), Staphylococcus aureus (#700699D), Streptococcus mutans (#25175D), Streptococcus pneumoniae (#6308D), Streptococcus pyogenes (#12344D), Candida albicans (#14053D). Escherichia coli gDNA was prepared in-house from an overnight culture of ATCC cells (#55151). Human gDNA was obtained from either the BioCrest production group (Cedar Creek, Tex.) or from BioChain (Hayward, Calif.; Human uterus genomic DNA, #D1234274-50). Mouse genomic DNA was obtained from Stratagene (#740009).

Quantitative PCR Assays

FullVelocity PCR assays were performed using reagents supplied by Stratagene's QPCR group in La Jolla, Calif. Assays were performed in 50 μl final volume. Sample/template to be amplified was added in 5 μl volume. When using the MX4000 PCR instrument (Stratagene), each assay contained final concentrations of: 15 mM Tris-HCl, pH 8.0, 50 mM KCl, 5.5 mM MgCl₂, 200 μM each of dATP, dGTP, and dCTP, 400 μM of dUTP, 8% glycerol, 1% DMSO, 4 ng/μl FEN-1 (Flap endonuclease), 0.05 U/μl V93R exonuclease-deficient Pfu polymerase, 30 nM ROX reference dye, 400 nM of each amplification primer (gbs1539-F and gbs1539-R for gbs1539 target, or GBSf2 and GBSr2 for CAMP-factor target), and 200 nM of the corresponding probe (gbs1539-P for gbs1539 target, or GBSp2 for CAMP-factor target). When included, the CAMP-factor internal control plasmid was present at 100 copies/PCR assay and internal control probe was present at 200 nM. On the MX4000, the cycling parameters were: 2 mm at 95° C., followed by 40 cycles of 10 sec at 95° C./30 sec at 60° C.

Alternatively, the above reaction conditions may be performed with 5 U of Pfu per reaction (0.1 U/μl) and 100 nM gbs1539-P. Gbs1539 internal control template may be included at 30 copies per reaction and PC-ICP (internal control probe) may present at 300 nM).

When adapting the gbs1539 assay for the fastest possible cycling conditions on the Mx3000p instrument (Stratagene), the gbs1539 probe concentration was reduced to 100 nM to increase the signal:noise. The finalized “fast” cycling parameters were: 2 min at 95° C., followed by 40 cycles of 1 sec at 95° C./18 sec at 60° C.

Clinical Isolates

The 82 clinical isolates of GBS were obtained from CPL (Austin, Tex.). At CPL, all samples were isolated from individual patient swabs, inoculated into selective media (TransVag broth), and grown overnight. The organisms were subcultured onto blood agar plates and grown overnight to produce isolated colonies. Single colonies from each sample were streaked onto fresh agar plates to produce homogeneous cultures for each of the 82 original patients. Plates were transported to BioCrest, and stored at −20° C. until use.

Each isolate was initially tested in PCR with the gbs1539 primer/probe set. A single colony was transferred to 250 p.l of Lysis Buffer (20 mM Tris, pH 8.0, 2 mM EDTA, 1.2% Triton X-100) and heated to 95° C. for 5 minutes. The sample was vortexed lor ˜15 seconds, and 5 μl was directly added to the PCR for testing. Isolates testing negative in the first PCR were retested with both the gbs1539 and CAMP-factor primer/probe sets.

Cloning of gbs1539 Internal Control

Arabidopsis thailana genomic DNA was purchased from BioChain (Hayworth, Calif.; # D4634310). The primers AthPC-GBS1539-F and AthPC-GBS1539-R primers (200 nM each, SEQ ID NOS:15 and 16), were used to amplify a fragment of the plastocyanin (PC) gene from Arabidopsis thaliana. The PCR product contains restriction cloning sites at the 5′ and 3′ ends, binding sites for the gbs1539 assay primers, and an internal sequence from the PC gene. The 180-bp product was excised from an agarose gel, purified, and digested with XhoI/BamHI. For long-term storage, the digested PCR fragment was cloned into pBluescript.

Primers:

AthPC-GBS1539-F (SEQ ID NO: 15):
TATACTCGAG [symbol] GGATGGTGACATGACAAAC
GGC
AthPC-GBS1539-R (SEQ ID NO: 16):
TATAGGATCC [symbol] TTAAGCAAGCATGGACAACT
ACAATAAGC

Cloning sites (XhoI/BamHI) are underlined, Gbs1539 primer binding sites are bolded/italicized, Internal control probe binding sequence is bolded/underlined
   Cloning of CAMP-Factor Internal Control

Arabidopsis thailana genomic DNA was purchased from BioChain (Hayworth, Calif.; # D4634310). Using primers AthPC-F and AthPC-R primers (200 nM each, Seq ID NOS:10 and 11), the plastocyanin gene fragment (SEQ ID NO:9) was amplified by PCR. The PCR fragment was excised from an agarose gel, purified, and digested with XhoI and BamHI restriction enzymes (Stratagene). The digested fragment was ligated into pBluescript SKII+ (Stratagene), and the product was transformed into XL-1 Blue E. coli (Stratagene). Colonies were screened for insert by PCR with GBSf2/GBSr2 primers. The sequence of the internal control insert (Seq ID NO:12) was verified by single-primer extension di-deoxy sequencing (Sequetech, Mountain View, Calif.).

### Example 2

### Primer and Probe Design

Two amplification primers and a single FAM-labeled probe were designed to specifically target gbs1539 (Seq ID NOs. 3-5). The primers were designed to amplify a 200 bp fragment. The melting temperatures of the primers are 59.8° C. for the forward primer and 60.7° C. for the reverse primer. The melting temperature of the probe is 64° C.

Along with the gbs1539 primer/probe set, a second set of oligonucleotides was designed to target the CAMP-factor gene (SEQ ID NO:2, 6-8). Due to the slightly lower G-C content of the CAMP-factor gene compared to gbs1539, it was difficult to find adequate primer-binding sites with average melting temperatures much greater than 58° C., even by lengthening the primers. Standard conditions for amplification using Stratagene's FullVelocity system are 60° C. for primer annealing. The chosen primers for CAMP-factor gene amplification melt at 57.7° C. for the forward primer and 59.1° C. for the reverse primer. The FAM labeled probe was designed with a melting temperature of 65° C. Based upon these parameters, the CAMP-factor target is predicted to be less robust in FullVelocity Q-PCR than the gbs1539 target due to the lower melting temperature of the primers.

### Example 3

### GBS (Group B Strep) Assay with gbs1539 Internal Control

The GBS Assay contains the FullVelocity QPCR reagents (Stratagene) at standard concentrations, gbs1539 forward and reverse primers (200 nM), gbs1539 probe (FAM-labeled) (100 nM), 180-bp internal control template (digested PCR product) (50-250 copies per reaction), Internal control probe (HEX-labeled) (300 nM), ROX reference dye (30 nM), Test sample (e.g. extract from clinical swab specimen).

The gbs1539 primer set amplifies both target GBS DNA (if present) and internal control template. The target GBS DNA is detected with the FAM channel, while the internal control is detected with the HEX channel.

### Example 4

### Sensitivity

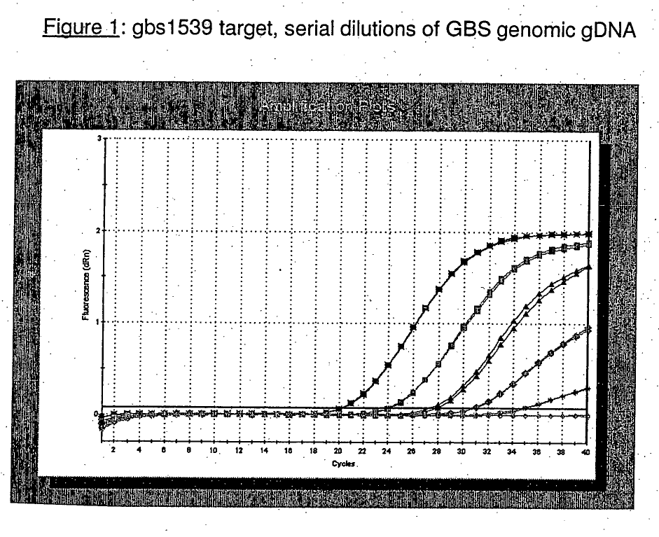

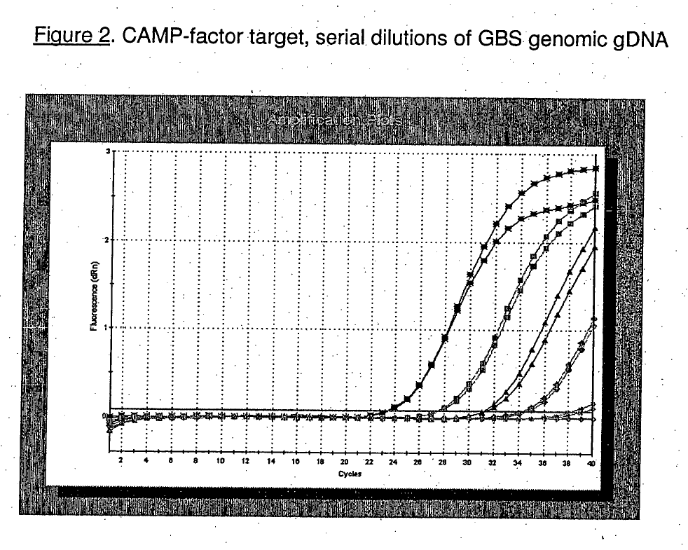

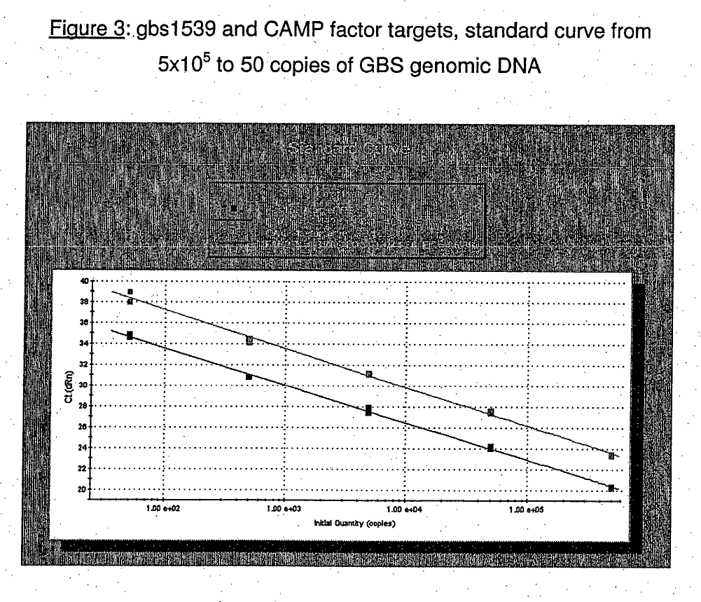

The two primer/probe sets were compared on the MX4000 real-time PCR platform by titrating purified GBS genomic DNA from 5×10⁵ to 50 input copies per PCR. The FullVelocity signal for the gbs1539 target was concentration dependent over 5 orders of magnitude with a linear fit R² value of 0.998 (FIGS. 1 and 3). In FIG. 1, concentrations of genomic DNA are as follows (from left-most blue line, in order): 5×10⁵, 5×10⁴, 5000, 500, and 50 copies/PCR. No-template control samples (orange line, open circles) contained TE only. Cycling parameters were as described in Materials and Methods. The trend of the data for the CAMP-factor target was nearly identical to gbs1539 (FIG. 2) between 5×10⁵ and 50 input copies. In FIG. 2, concentrations of genomic DNA are as follows (from left-most blue line, in order): 5×10⁵, 5×10⁴, 5000, 500, and 50 copies/PCR. No-template control samples (orange line, open circles) contained TE only. Cycling parameters were as described in Materials and Methods. In FIG. 3, grey line=gbs1539; black line ═CAMP-factor. Threshold value for both targets were set at 0.075. The variation of the data points from the best line (R²) is 0.998 for gbs1539 and 0.996 for CAMP-factor. On average, the gbs 1539 signal is detected ˜3.5 cycles earlier than CAMP-factor. The efficiency of amplification was also slightly higher for gbs1539 (90.9%) than CAMP-factor (86.3%). The correlation coefficient (R² value) was 0.996 (FIG. 3), with the 50-copy sample producing the greatest variation between duplicates. The major difference between the two assays, however, was that the threshold cycle (Ct) for the gbs1539 target was reached, on average, ˜3.5 cycles earlier than for the CAMP-factor target across the entire range of genomic DNA concentrations. As a result, the gbs1539 assay should require shorter cycling time than the CAMP-factor assay to produce a signal for a given target DNA concentration.

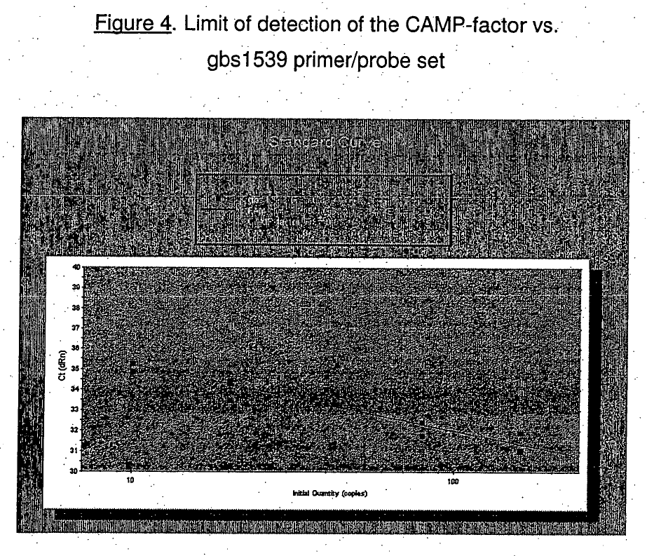

To determine the absolute limit of detection of the assays at low input genomic DNA copy numbers, the comparison was repeated by testing serial dilutions of 1:2, from 160 copies to 2.5 copies. Data at this low concentration range was not strictly quantitative due to stochastic variations between samples. The gbs1539 target was reproducibly detected down to 10 copies of genomic DNA as determined by testing in triplicate in two independent experiments (FIG. 4). In contrast, the CAMP-factor target could not be detected reproducibly below 20 copies. The variance in replicates was greater for CAMP-factor as evidenced by lower R² values (FIG. 4) in comparison to gbs1539. Therefore, the gbs1539 assay is more reliable at lower copy numbers and slightly more sensitive than the CAMP-factor assay. In FIG. 4, samples were tested in triplicate. Two independent experiments produced similar results, but only one data set is shown here. The CAMP-factor assay could not detect all of the replicates for the “10 copy” samples, so the points have been omitted to better estimate the linear fit of the data. The R² value for the gbs1539 assay was 0.869, and the R² value for the CAMP-factor assay was 0.838. The line plotted from the average of the data points (not shown) has an R² value of 0.943 for the gbs1539 assay and 0.916 for the CAMP-factor assay.

### Example 5

### Specificity

The specificity of detection was analyzed by comparing each GBS primer/probe set in assays containing genomic DNA from a range of bacterial or fungal species. Organisms were chosen that are likely to be present in vaginal/rectal specimens, as well as those that are evolutionarily related to Streptococcus agalactiae. In particular, genomic DNAs from Enterococcus faecalis, Lactococcus lactis, Listeria monocytogenes, Staphylococcus aureus, Streptococcus mutans, Streptococcus pneumoniae, Streptococcus pyogenes, Escherichia coli, and Candida albicans were all tested with both primer/probe sets. In addition, human and mouse genomic DNAs were tested to determine if non-specific primer/probe interactions could result in false-positive signals. Even when non-specific bacteria/fungal DNA at levels of 50 to 500 times that of S. agalciatiae was added to the PCR, only S. agalacitiae genomic DNA produced a signal (data not shown). Likewise, 10-100 ng of human or mouse gDNA did not give rise to a signal with either primer/probe set. These data indicate the specificity of the CAMP-factor and gbs1539 targets.

To test the universality of the gbs1539 target across individual clinical specimens, 82 isolates from 82 different patients were tested for gbs1539 (see Materials and Methods). Patient swabs were subcultured in selective media and streaked onto blood agar plates (CPL, Austin, Tex.). Individual colonies were subcultured again onto fresh blood agar plates prior to transport and storage at 4° C. Single colonies from each of the 82 plates were lysed and tested directly in the PCR. 80 out of 82 samples produced robust signals in the initial screen using the gbs1539 primer/probe set (Table I). The 2 negative samples were retested with both the gbs1539 primer/probe set and the CAMP-factor primer/probe set. Both samples were again negative for gbs1539, and also negative for CAMP-factor. Genomic DNA from both negative samples was prepared a second time, and retested in PCR with both primer/probe sets. One of the negative samples reverted to positive status when screened for both the gbs1539 and CAMP-factor targets. However, the other sample remained negative for both gbs1539 and CAMP-factor.

In summary, 81 of 82 clinical isolates contain DNA for gbs1539 that is detected by the primer/probe set (Table I). The single negative isolate was also negative for CAMP-factor, even after repeated testing. Due to the ubiquitous nature of the CAMP-factor gene (Podbielski et al., 1994, Molecular characterization of the cfb gene encoding group B streptococcal CAMP-factor. Med Microbiol Immunol. 183:239-56), it is likely that inhibitors were present that caused PCR failure with both targets, or the isolate was not GBS. Therefore, we conclude that the gbs1539 target is conserved among a vast majority, if not all, of clinically-derived Group B Streptococcus isolates. Further testing with a greater number of isolates from different geographical areas is required to determine a definitive degree of conservation of the gbs1539 gene.

### Summary of gbs1539 Sensitivity Testing

### 82 Clinical Isolates Tested

| | First trial (Pos | Retest | Reprep/retest | Totals (Pos |
|---|---|---|---|---|
| | samples/total | negative | negative | samples/total |
| Target | tested) | isolates | isolates | tested) |
| gbs1539 | 80*/82 | 0/2 | 1/2 | 81/82 |
| CAMP factor | Not tested | 0/2 | 1/2 | 1/2 |

*Cts range from 17-22

### Example 6

### Increased Assay Speed on the Mx300p

Since the objective of developing a real-time assay for GBS colonization diagnosis is to provide results to the mother as quickly as possible during the time course of labor, the gbs1539 assay was tested for increased speed on the Mx3000p real-time PCR instrument (Stratagene). The Mx3000p has quicker ramping times between temperatures than the MX4000 and is therefore more suited to obtaining quick results. The quickest minimal cycling parameters (1 sec denaturation and 18 sec annealing/extension) were tested using the gbs1539 primer set with the gbs1539 probe. Due to differences in the background signal between the MX4000 and Mx3000p, the probe concentration was decreased to 100 nM when using the Mx3000p to increase the signal-to-noise ratio (see Materials and Methods).

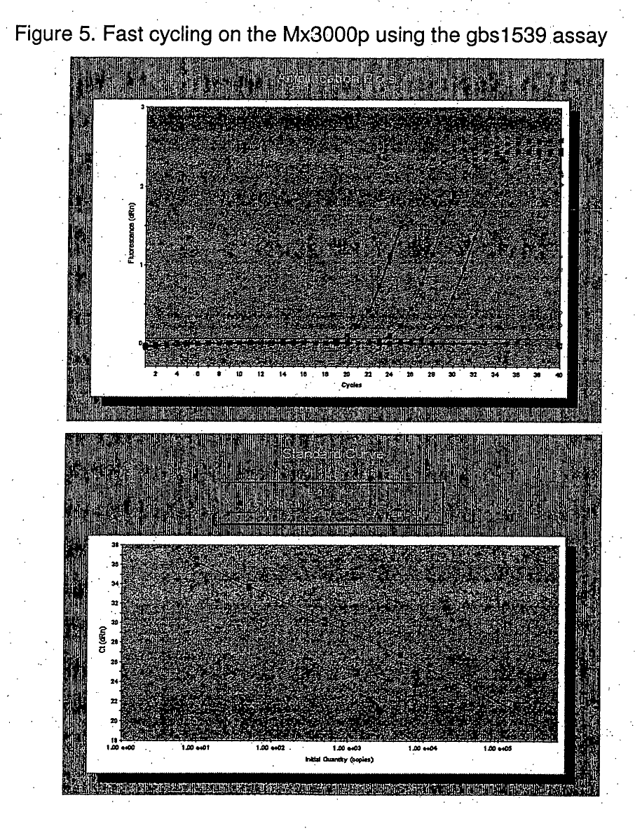

While the total cycling time on the MX4000 is just over 1 hour, cycling time could be reduced to 46 minutes using the faster protocol combined with faster ramping times on the Mx3000p. The sensitivity of the assay was the same as on the MX4000 (10 copies), and the signal was concentration-dependent with a linear fit R² value of 0.998 from 5×10⁵ to 10 copies of GBS genomic DNA (FIG. 5). In FIG. 5, cycling parameters are: 2 min at 95° C., followed by 40 cycles of 1 sec at 95° C./18 sec at 60° C. for a total of 46 minutes. The order of gDNA samples in the top panel from left to right is 5×10⁵, 5×10⁴, 5000, 500, 50 and 10. The R² value for the standard curve (bottom) is 0.998. The open squares represent no-template controls (TE only).

Therefore, using the gbs1539 primer/probe set, results can be obtain in just over 45 minutes, similar to the claims made by the IDI-Strep B assay manual. Including sample preparation time, diagnosis could be made in under 1 hour, based upon preliminary data, even when using spin cup sample purification (not shown).

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

What is claimed is:

1. A method of detecting the presence of a Group B Streptococcus (GBS bacterium) in a sample, comprising:
(a) contacting said sample with a primer which hybridizes to the sequence of SEQ ID NO:1 or its complementary sequence thereof under conditions permitting the production of an extension product from said primer; and
(b) detecting the presence of said extension product, wherein said presence of said extension product is indicative of the presence of a GSB bacterium in said sample.

2. The method of claim 1, wherein said extension product is produced by a polymerase chain reaction (PCR).

3. The method of claim 1, wherein said primer comprises a sequence of SEQ ID NO:3 or SEQ ID NO:4 or a complementary sequence thereof.

4. The method of claim 1, wherein said step (a) comprising contacting said sample with a pair of primers, wherein at least one primer comprises a sequence of SEQ ID NO:3 or SEQ ID NO:4 or a complementary sequence thereof.

5. The method of claim 1, further comprising a labeled probe in step (a), wherein said probe hybridizes to said extension product and said hybridization generates a detectable signal which is indicative of the presence of a GSB bacterium in said sample.

6. The method of claim 5, wherein said labeled probe comprises a sequence of SEQ ID NO:5 or a complementary sequence thereof.

7. The method of claim 5, wherein said probe is labeled with a detectable label and said hybridization generates a detectable-signal which is indicative of the presence of a GBS bacterium in said sample.

8. The method of claim 7, wherein said detectable label is a fluorescent label.

9. The method of claim 1, wherein said sample is obtained from an individual suspected of being infected with GBS.

10. An isolated oligonucleotide comprising a sequence selected from the group consisting of SEQ ID NOs. 3-5 and their complementary sequences thereof.

11. The isolated oligonucleotide of claim 10, wherein said oligonucleotide is 8-100 nucleotides in length.

12. The isolated oligonucleotide of claim 11, wherein said oligonucleotide is 15-50 nucleotides in length.

13. An isolated polynucleotide comprising a sequence of SEQ ID NO:9 or SEQ ID NO:14.

14. A pair of isolated oligonucleotides comprising a first oligonucleotide and a second oligonucleotide, wherein said first oligonucleotide comprises the sequence of SEQ ID NO:3 or its complementary sequence thereof and said second oligonucleotide comprises the sequence of SEQ ID NO:4 or its complementary sequence thereof.

15. A composition comprising an oligonucleotide comprising a sequence selected from the group consisting of SEQ ID NOs. 3-5 and their complementary sequences thereof.

16. The composition of claim 15, wherein said oligonucleotide is 8-100 nucleotides in length.

17. The composition of claim 16, wherein said oligonucleotide is 15-50 nucleotides in length.

18. The composition of claim 15, further comprising a reagent selected from the group consisting of: a DNA polymerase, a control DNA, a control primer, and a deoxynucleotide triphosphate (dNTP).

19. A kit comprising an oligonucleotide comprising a sequence selected from the group consisting of SEQ ID NOs. 3-5 and their complementary sequences thereof, and packing materials therefore.

20. The kit of claim 19, further comprising a reagent selected from the group consisting of a DNA polymerase, a control DNA, a control primer, and a dNTP.
